(19) United States
(12) Patent Application Publication
(10) Pub. No.: US 2013/0226200 A1
(43) Pub. Date: Aug. 29, 2013

(54) CLIP APPLIER

(71) Applicant: BOSTON SCIENTIFIC SCIMED, INC., (US)

(72) Inventors: Gary KAPPEL, Acton, MA (US); Paul Smith, Smithfield, RI (US); Barry Weitzner, Acton, MA (US)

(73) Assignee: BOSTON SCIENTIFIC SCIMED, INC., Maple Grove, MN (US)

(21) Appl. No.: 13/779,086

(22) Filed: Feb. 27, 2013

Related U.S. Application Data
(60) Provisional application No. 61/604,012, filed on Feb. 28, 2012.

(51) Int. Cl. A61B 17/128; A61B 17/122

(52) U.S. Cl. CPC A61B 17/128; A61B 17/122
USPC 606/142; 606/158

(57) ABSTRACT

A system for performing a medical operation includes an end effector having first and second arms, each extending from a distal end to a proximal end, the proximal ends being connected to one another, the end effector being movable between a closed configuration wherein the first and second arms are separated from one another by a first distance and an open configuration wherein the first and second arms are separated from one another by a second distance greater than the first distance, a distance between the first and second arms being substantially constant from the proximal ends to the distal ends, wherein the end effector is biased to the open configuration. A first plate element extends from a first surface configured to engage a first wall of the first arm to a second surface configured to contact target tissue in an operative configuration. A second plate element extends from a first surface configured to engage a first wall of the second jaw to a second surface configured to contact target tissue in an operative configuration.

### PRIORITY CLAIM

This present application claims the priority to the U.S. Provisional Application Ser. No. 61/604,012, entitled “Clip Applier” filed on Feb. 28, 2012. The specification of the above-identified application is incorporated herewith by reference.

### BACKGROUND

Surgical procedures often require the clamping of vessels to prevent the flow of fluids therethrough. Although clipping devices have been employed in controlling internal bleeding (e.g., from surgical wounds or naturally occurring damage) these clips are generally unsuitable to clamp vessels to prevent fluid flow therethrough.

### SUMMARY OF THE INVENTION

The present invention relates to a system for performing a medical operation which includes an end effector having first and second arms, each extending from a distal end to a proximal end, the proximal ends at the arms being connected to one another, the end effector being movable between a closed configuration wherein the first and second arms are separated from one another by a first distance and an open configuration wherein the first and second arms are separated from one another by a second distance greater than the first distance, a distance between the first and second arms being substantially constant from the proximal ends to the distal ends, wherein the end effector is biased to the open configuration. A first plate element extends from a first surface configured to removably engage a first wall of the first arm to a second surface configured to contact target tissue in an operative configuration. A second plate element extends from a first surface configured to removably engage a first wall of the second jaw to a second surface configured to contact target tissue in an operative configuration.

### BRIEF DESCRIPTION OF THE DRAWINGS

FIG. 1 shows a partially exploded view of a device according to a first exemplary embodiment of the present invention;

FIG. 2 shows a perspective view of the device of FIG. 1 in an assembled configuration;

FIG. 3 shows a side view of the device of FIG. 1 in a first operative configuration;

FIG. 4 shows a side view of the device of FIG. 1 in a second operative configuration;

FIG. 5 shows another perspective view of the device of FIG. 1 in the second operative configuration;

FIG. 6 shows a perspective view of the device of FIG. 1 in a first operative configuration;

FIG. 7 shows a perspective view of the device of FIG. 1 in a second operative configuration;

FIG. 8 shows a perspective view of a device according to a first alternate embodiment of the invention;

FIG. 9 shows a first perspective view of a device according to a second alternate embodiment of the invention;

FIG. 10 shows a second perspective view of the device of FIG. 9;

FIG. 11 shows a third perspective view of the device of FIG. 9;

FIG. 12 shows a partial cross-sectional view of the device of FIG. 9;

FIG. 13 shows a perspective view of a device according to a third alternate embodiment of the invention;

FIG. 14 shows a perspective view of a device according to a fourth alternate embodiment of the invention;

FIG. 15 shows a perspective view of a device according to a fifth alternate embodiment of the invention;

FIG. 16 shows a first perspective view of a device according to a sixth alternate embodiment of the invention;

FIG. 17 shows a second perspective view of the device of FIG. 16;

FIG. 18 shows a first perspective view of a device according to a seventh alternate embodiment of the invention;

FIG. 19 shows a second perspective view of the device of FIG. 18;

FIG. 20 shows a first perspective view of a device according to an eight alternate embodiment of the invention; and

FIG. 21 shows a second perspective view of the device of FIG. 20.

### DETAILED DESCRIPTION

The present invention may be further understood with reference to the following description and the appended drawings, wherein like elements are referred to with like reference numerals. The present invention relates to a clip for use in sealing lumens of body vessels and/or to an adaptor plate configured for placement over arms of a known hemostasis clip to configure the clip for use in sealing a body lumen, vessel, duct or organ. The clips according to the invention are configured to apply a substantially constant constrictive pressure over a target portion of tissue while minimizing trauma thereto to substantially seal a vessel within the tissue. In an exemplary embodiment, the adaptor plates are configured so that, when added to an existing hemostatic clip, a surface area over which arms of the clip contact tissue is expanded to spread a force drawing the clip arms closed over a wider surface area of tissue—e.g., to prevent fluid flow through a target vessel, as will be described in greater detail later on. Exemplary adaptor plates according to the invention may attach to the clip arms via one or more of a snap-fit, tongue and groove mechanism, hinge or by any other means and apparatuses known in the art. Thus, a clip according to the invention or a known clip including exemplary adaptor plates may be deployed over the target vessel using a standard hemostatic clipping device as known in the art. It is noted that although the exemplary device according to the invention has been described with respect to clipping devices, the adaptor plates may also be employed in conjunction with devices designed to perform any of a variety of endoscopic procedures. Furthermore, the adaptor plates may be used with any clipping device without deviating from the scope of the invention. It should be noted that the terms “proximal” and “distal,” as used herein, are intended to refer to a direct toward (proximal) and away from (distal) a user of the device.

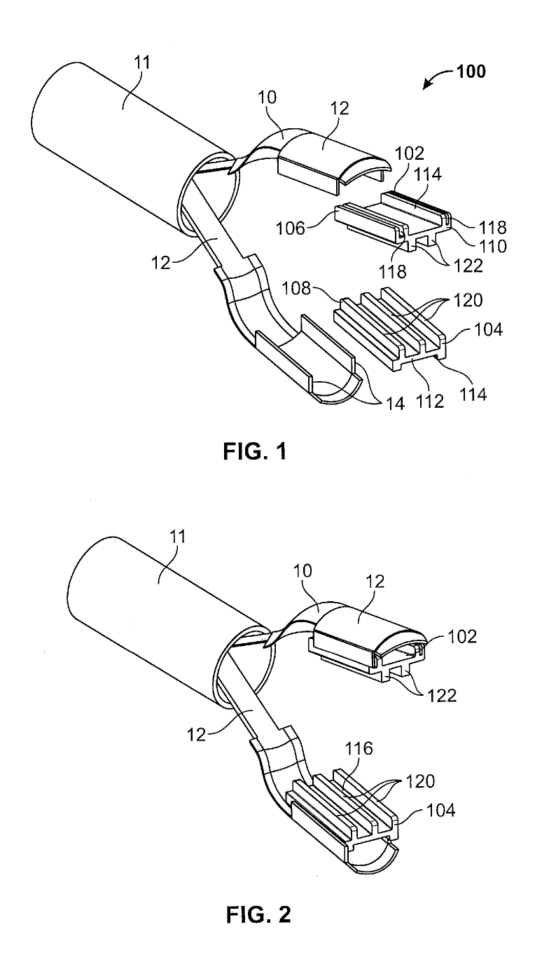

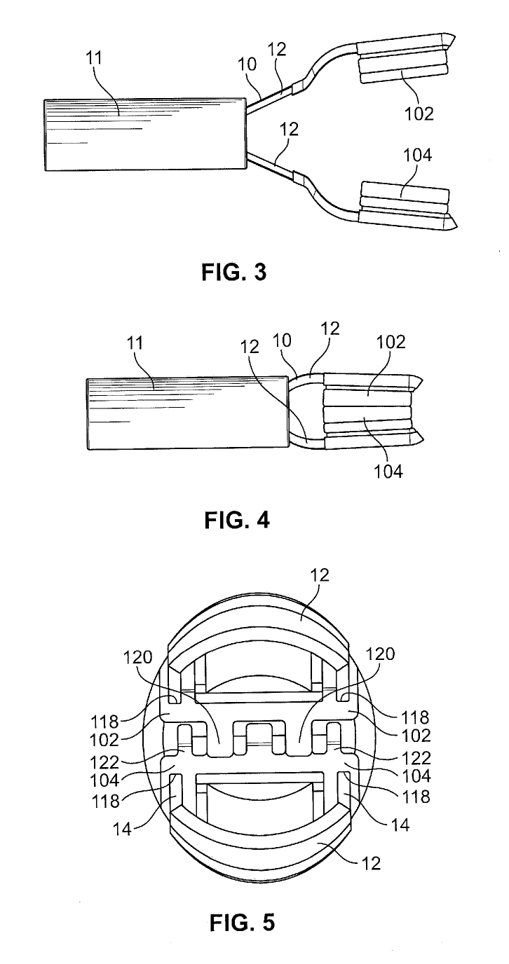

As shown in FIGS. 1-6, a device 100 according to an exemplary embodiment of the invention comprises first and second adaptor plates 102, 104 extending longitudinally from proximal ends 106, 108 to distal ends 110, 112, respectively. An axial length of each of the adaptor plates 102, 104 may be selected to substantially conform to a length of clip arms 12 of a hemostasis clip 10 to which they are removably attached. In another embodiment, as shown in FIG. 15, a distal end of the adaptor plate may extend distally beyond a distal end of the clip (e.g., to grasp a larger artery therebetween), as will be described in greater detail later on. In an exemplary embodiment, the clip arms 12 are biased toward an open configuration and held in a closed configuration by engagement with the capsule 11 or walls of a magazine (not shown). The hemostasis clip and related delivery device may be formed, for example, substantially similarly to the device disclosed in U.S. Pat. No. 7,494,461 entitled “Through the Scope Tension Member Release Clip” filed on Sep. 30, 2003, hereinafter referred to as the '461 patent, the entire disclosure of which is incorporated herein by reference. Whereas conventional clips are configured to grasp tissue only via sharpened distal portions of each clip arm, the exemplary clip 10 according to the invention (or a conventional clip modified as described below) is configured so that substantially an entire length of each of the arms 12 applies an even pressure to tissue grasped between the arms 12. Specifically, a surface of each of the clip arms 12 facing the other arm 12 is substantially flat along a tissue engaging length thereof and this flat tissue engaging surface may also extend across an entire width of the arms 12 to diffuse the gripping force substantially evenly over a wide area of tissue. The term “flat” as used herein is not restricted to a single plane. Rather, it is intended to define a surface which is substantially in contact with tissue from one end of the surface to the other (e.g., along a full length of the clip).

Each of the adaptor plates 102, 104 comprises an arm-gripping surface 114 and a tissue-gripping surface 116. The arm-gripping surface 114 may also include one or more grooves 118 adjacent lateral edges thereof and configured to engage with extensions 114 extending from lateral edges of the clip arms 12 and locked thereto via a snap-fit engagement. As would be understood by those skilled in the art, the arm-gripping surface 114 may be formed with any other configuration selected to conform to a shape and size of the clip arms 12 without deviating from the scope of the invention. For example, the arm-gripping surface may include any combination of tabs, slots, holes, bumps, etc. without deviating from the scope of the invention. In an exemplary embodiment, a frictional-fit engagement of the extensions 14 with the grooves 118 prevents the adaptor plates 102, 104 from sliding or moving out of engagement with the clip arms 12. The adaptor plates 102, 104 may be configured to prevent sliding thereof proximally or distally out of alignment with the clip arms 12. In one embodiment, one or both of a proximal and distal end of one or both grooves 118 may comprise a stop (not shown) to prevent sliding of the adaptor plates 102, 104 from a desired position. In another embodiment, the adaptor plates 102, 104 may be secured to the clip arms 12 by one or more of a snap-fit, detent, mating pins and holes, interference fit, etc. As would also be understood by those skilled in the art, the adaptor plates 102, 104 are preferably designed to conform to a length and width of a selected portion of each of the arms 12.

As shown in FIGS. 1-6, tissue-gripping surfaces 116 of the adaptor plates 102, 104 according to a first exemplary embodiment of the invention comprise one or more longitudinal protrusions 120, 122 extending substantially parallel to longitudinal axes of the adaptors 102, 104, respectively. As those skilled in the art will understand, the protrusions 120, 122 on opposing adaptor plates 102, 104 are preferably configured to interlink with one another in a closed configuration to aid in occlusion of a target vessel, as will be described in greater detail with respect to the exemplary method. Specifically, the first adaptor plate 102 according to this embodiment comprises two protrusions 120 while the second adaptor plate 104 comprises three protrusions 122. The protrusions 120 are received in grooves defined between protrusions 122 so that flat portions of the tissue-gripping surfaces 116 are brought into close contact with one another when a clip including the adaptor plates 102, 104 is closed. As those skilled in the art will understand, this cooperation between the protrusions 122 and grooves of the adaptor plates 102, 104 forces the walls of a target vessel captured therebetween into a tortuous path which enhances a sealing effect on the vessel. It is further noted that any number of protrusions 120, 122 may be provided on the first and second adaptor plates 102, 104 in any orientation without deviating from the scope of the invention. Furthermore, the protrusions 120, 122 may be provided in any shape (e.g., diagonal, zig-zag, curved, “S” shaped, serpentine, etc.) without deviating from the scope of the invention. The protrusions 120, 122 may also be in the form of bumps, spikes, teeth, intermittent tabs, etc. In another embodiment, the protrusions may be provided over only a distal portion of one or both of the plates 102, 104 and may extend perpendicular to a longitudinal axis of the plates 102, 104.

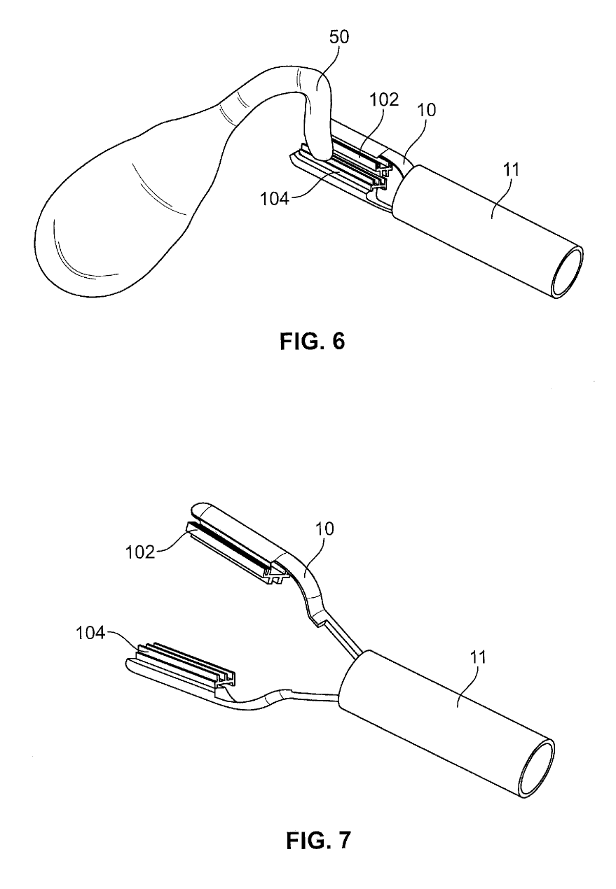

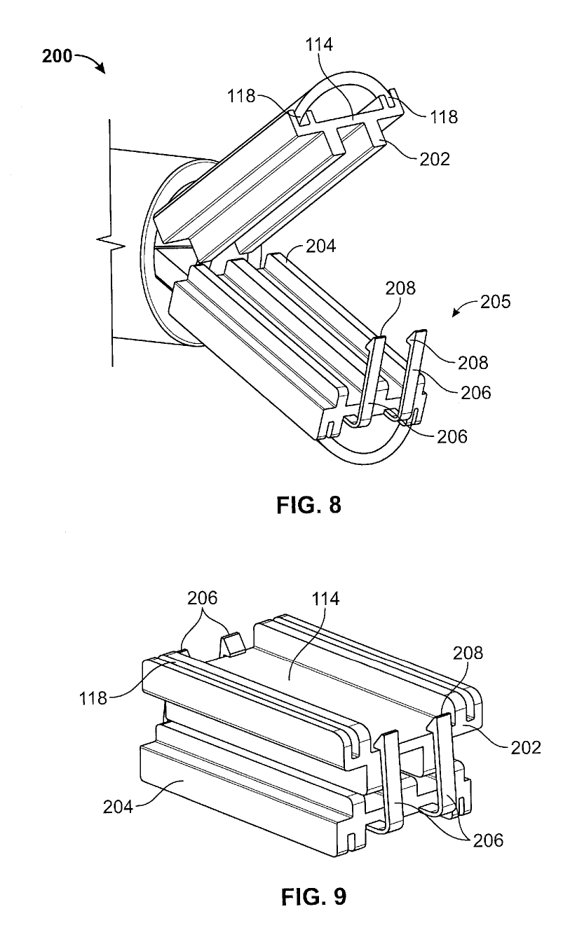

FIGS. 6-7 depict the device 100 in an operative configuration sealing a portion of a cystic duct 50 although those skilled in the art will understand that the device 100 may be deployed to prevent flow through any suitably sized vessel such as, for example, a blood vessel, lumen, duct or organ. FIG. 6 depicts the device 100 in an open configuration prior to placement thereof over the cystic duct 50. FIG. 7 depicts the device 100 in a closed, sealing configuration over the cystic duct 50. In accordance with an exemplary method according to the invention, a proximal portion of the clip 10 is housed within the capsule 11 and is pre-loaded into a single-use delivery device (not shown). For example, the device 100 may include, as described in greater detail in the '461 patent, a clip 10 removably connected to a control wire (not shown) extending through a delivery device. In an operative configuration, the control wire is actuated to cause movement of the clip 10 distally out of a distal end of the delivery device. As the clip 10 is moved out of the delivery device, the clip arms 12 are moved to the open configuration (e.g., by a natural bias of the clip arms 12), and positioned as desired across a cystic duct 50, as shown in FIG. 6. Once the clip 10 has been positioned as desired across the cystic duct 50, the control wire is drawn proximally, retracting the clip 10 partially into the capsule 11 so that contact between the capsule 11 and the arms 12 of the clip 10 draws the arms 12 to the closed configuration over the cystic duct 50. If the tension on the control wire is increased beyond a threshold level, a connection between the control wire and the clip 10 is released locking the clip 10 closed over the target tissue and separating the clip 10 and the capsule 11 from the delivery device, for example, as described in the '461 patent. With the clip 10 closed over, for example, the cystic duct 50, pressure is applied substantially evenly across the width of walls of the cystic duct 50 by the adaptor plates 102, 104 minimizing force concentration on any portion of the cystic duct 50 to reduce trauma thereto. At the same time, the substantially even application of force across the width of the duct 50 can help prevent fluid flow therethrough by ensuring that opposite portions of the walls of the duct 50 are brought into contact with one another across the extent of the lumen of the duct 50. As those skilled in the art will understand, the clip 10 and adaptor plates 102, 104 may be formed of any suitable biocompatible, absorbable material (e.g., PLA suture material) configured to be absorbed after a predetermined period of time (e.g., 3 days, 1 week, 1 month, etc.), thus eliminating the need for removal of the clip 10 from the body as the clip will naturally release and permit fluid flow through the lumen after a predetermined time has elapsed. The clip 10 and adaptor plates 102, 104 may be configured to permanently or temporarily occlude an artery, vessel, duct, etc. In one embodiment, a material of the clip 10 and adaptor plates 102, 104 may be configured to weaken over time to allow the vessel to resume normal functioning after a selected period of time has elapsed (e.g., a period of time selected to be less than a time required to permanently impair the function of the clipped tissue). In another embodiment, the clip 10 and adaptor plates 102, 104 may be formed of a metal (e.g., stainless steel, titanium)or a plastic (e.g., PEEK). In another embodiment, the clip 10 and adaptor plates 102, 104 may be surgically removed from the patient to open the vessel to flow.

As shown in FIG. 8, a device 200 according to a first alternate embodiment of the invention may be formed substantially similarly to the device 100 and comprises first and second adaptor plates 202, 204. Similar to the device 100, the device 200 comprises a pair of grooves 118 adjacent lateral edges thereof configured to receive the extensions 14 on lateral portions of the clip arms 12. One or both distal ends 110, 112 of the adaptor plates 202, 204 comprise a mechanism 205 configured to lock the adaptor plates 202, 204 in the closed configuration. The mechanism 205 may comprise one or more transverse extensions 206 extending away from the adaptor plate 204. The extensions 206 may be a separate mechanism from the clip arms or may be formed integral to one or both of the clip arms. In one embodiment, the extensions 206 are biased to a configuration extending substantially orthogonal to a longitudinal axis of the arms 12. The extensions 206 comprise tabs 208 at free ends thereof, the tabs 208 configured to lockingly engaging the clip arm gripping surface 114 of the opposing plate 202 when the clip arms 12 are moved to a tissue gripping configuration with tissue captured therebetween. That is, a length of the extensions 206 is selected to permit the tabs 208 to lockingly engage the opposing plate 202 when the adaptor plates 202, 204 are separated from one another by a distance selected to permit application of a target pressure to tissue captured therebetween, as shown in FIG. 9. As described in greater detail in the '461 patent, the clip 10 may assume a first partially open configuration when housed in the delivery device. This configuration prevents movement of the tabs 208 to the locked configuration prior to deployment. It is noted that although the device 200 is depicted with two extensions 206, any number of extensions 206 may be employed without deviating from the scope of the invention.

In further embodiment, the locking mechanism may be situated such that tabs 208 insert through the adaptor plate and clip arm opposite the clip arm from which the extension 206 extends. In some embodiment, extension 206 comprises a ratcheting surface to allow the clip to be compressed at varying distances between the arms. Increased compression on the clip arms will bring them closer together and the ratcheting surface will maintain the selected distance.

In an operative configuration, as the clip 10 is moved to a closed configuration and the arms 12 are drawn together, the first adaptor plate 202 moves radially inward past the tab 208, thereby lockingly seating the tab 208 against the clip arm-gripping surface 114. As those skilled in the art will understand, engagement of the tab 208 with the arm-gripping surface 114 locks the adaptor plates 202, 204 in the closed configuration and prevents the clip 10 from returning to the open configuration. Although the device 200 has been described with respect to a locking tab, any other locking mechanism may be employed without deviating from the scope of the invention, including, but not limited to a ratcheting snap-mechanism.

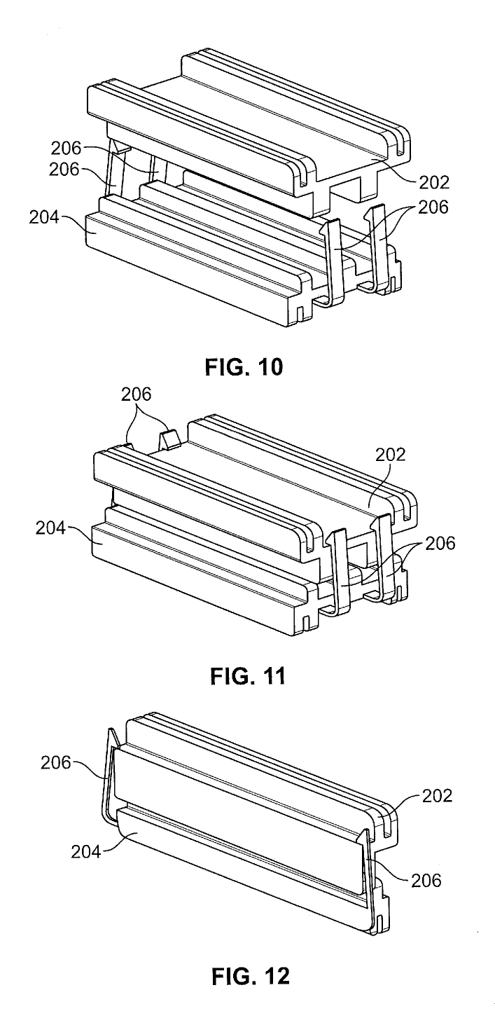

As shown more clearly in FIGS. 9-12, the adaptor plate 204 may also comprise a pair of extensions 206 on each of the proximal and distal ends thereof. Each of the pair of extensions 206 operates in a manner similar to that disclosed above. In one embodiment of the invention, the device of FIGS. 9-12 may be used with a clip (not shown) having clip arms that extend substantially parallel to one another in both the open and closed configuration. Specifically, the clip (not shown) may be configured so that the adaptor plates 202, 204 remain substantially parallel to one another to permit the extensions 206 to engage the clip arm-gripping surface of an opposing adaptor plate at the same time. It is noted, however, that the embodiment of FIGS. 9-12 may also be used with the clip of FIG. 8, wherein the proximal extensions 206 are only moved to a locked configuration upon deployment of the adaptor plates 202, 204 from the clip 10.

The adaptor plates 202, 204 may be attached to one another by one or more flexible hinges or any other attachment mechanism known in the art. The hinge may permit pivotal movement of the plates 202, 204 and may be biased to first partially open configuration as described in greater detail earlier. In another embodiment, the hinge may be a living hinge.

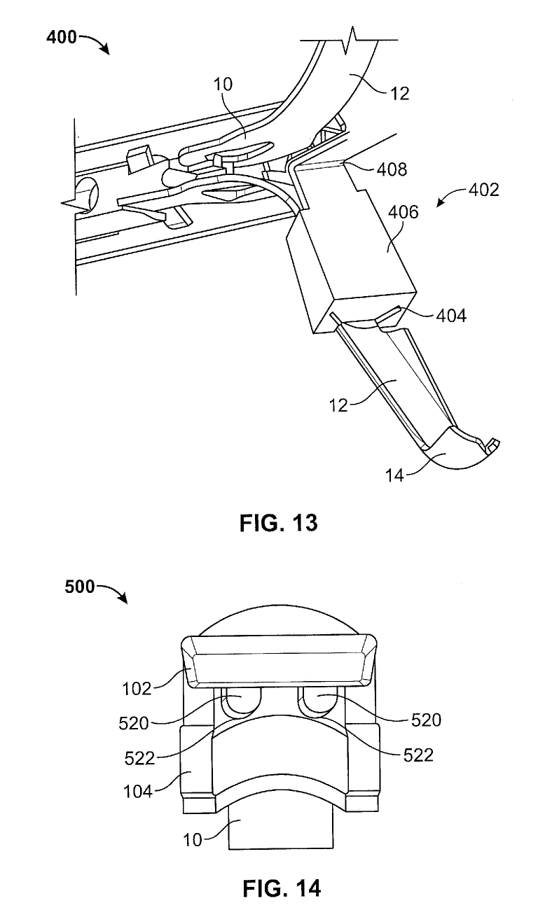

FIG. 13 depicts a device 400 according to another embodiment of the invention wherein the clip 10 comprises an insert 402 positioned over one clip arm 12. The insert 402 may be provided in a kit provided to an end-user. The insert 402 may be provided in a cartridge or other device to aid in insertion thereof over the clip 10 pre-operatively. A flared portion 14 of the clip arm 12 prevents the insert 402 from sliding off of the clip 10. In another embodiment, the insert 402 may be preloaded onto the clip 10 during manufacturing. The insert 402 comprises an opening 404 slidably received over the arm 12, the insert 402 being slidable along a length of the arm 12. The insert 402 may comprise a first adaptor plate 406 and a second adaptor plate (not shown) configured to apply a pressure to a vessel in an operative configuration, the arms being connected to one another by a living hinge 408. In another embodiment, the second adaptor plate (not shown) may also comprise an opening (not shown) configured to permit insertion thereof over the other clip arm 12. In yet another embodiment, the insert 402 may comprise only the first adaptor plate 406 configured so that a target vessel or other target tissue is captured between the first adaptor plate 406 and an opposing clip arm 12. As also shown in FIG. 13, the adaptor plate 406 may be formed with an axial length smaller than an axial length of the clip arms 12. As those skilled in the art will understand, this configuration permits installation of the adaptor plate 406 over a flared edge of the clip arm 12. The adaptor plate 406 is then advanced proximally over the clip arm 12 to a retaining configuration. The first and second adaptor plates disclosed herein are not restricted to the disclosed means of attachment to the clip arms 12. Specifically, the first and second plates may be secured to the clip arms 12 by any other means, including, but not limited to, press-fit, snap-fit, screwing via a threaded post and complementary hole, magnets, or may be inserted over the clip arms via openings extending through the first and second plates.

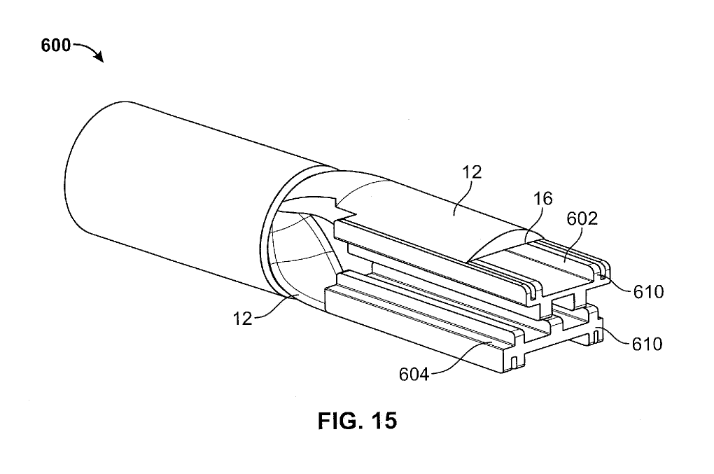

FIG. 14 depicts a device 500 according to another embodiment of the invention, the device 500 being formed substantially similar to the device 100, wherein like elements have been referenced with like reference numerals. The device 500 differs from the device 100 in that the first adaptor plate 102 comprises protrusions 520 formed similar to the protrusions 120 while the second adaptor plate 104 comprises recesses 522 configured to seat the protrusions 520 therein. As those skilled in the art will understand, this configuration aids in the application of a constrictive pressure to a target vessel constrained therebetween.

FIG. 15 depicts a device 600 according to another embodiment of the invention. The device 600 is formed substantially similarly to the device 100 of FIGS. 1-6 with the exception of a distal end 610 of adaptor plates 602, 604 extending distally beyond a distal end 16 of the clip arms 12. As those skilled in the art will understand, this embodiment permits the gripping of a larger area by the adaptor plates 602, 604 with, for example, a first portion of a target vessel constricted by the clip arms 12 and a remainder thereof constricted by the adaptor plates 602, 604. The adaptor plates 602, 604 may be provided with any of a range of stops to limit axial sliding thereof relative to the clip arms 12 within a desired range, as described in greater detail earlier.

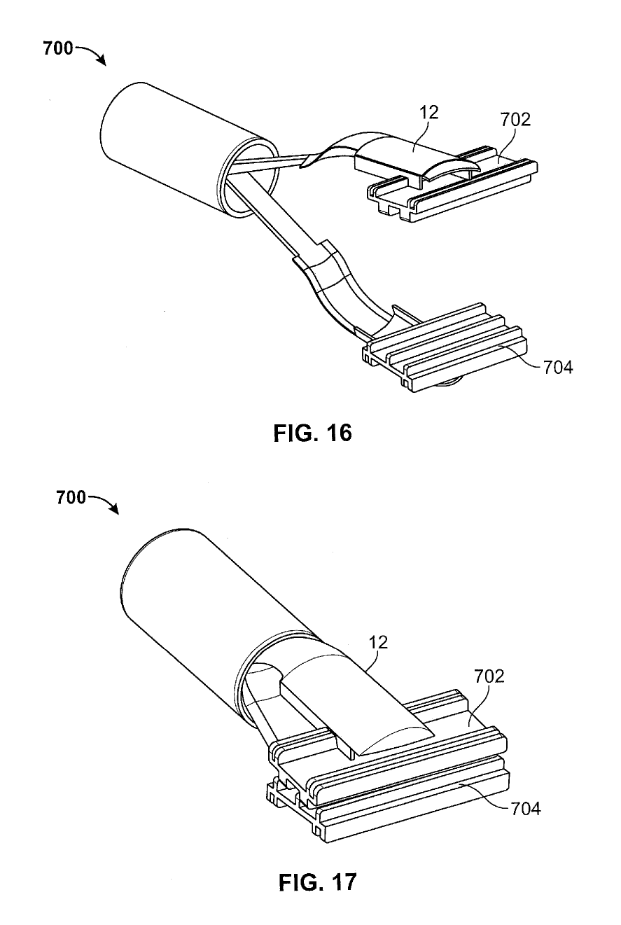

FIGS. 16-17 depicts a device 700 according to another embodiment of the invention, the device 700 being formed substantially similar to the device 100. The adaptor plates 702, 704 are configured to be rotatable relative to the clip arms 12. In an exemplary embodiment, the adaptor plates 702, 704 may be positioned at any angle relative to a longitudinal axis of the clip arms 12. Specifically, the adaptor plates 702, 704 may be attached to the clip arms by an attachment means (not shown) defining a point of rotation of the clip arms 12. In the embodiment shown, the adaptor plates 702, 704 are rotated to a position extending approximately 90 degrees relative to the longitudinal axis of the clip arms 12. The device 700 may comprise a locking mechanism (not shown) permitting locking of the adaptor plates 702, 704 in the desired angular configuration. The adaptor plates 702, 704 may be connected to a break-away pull cable (not shown) or another mechanism configured to control an angle thereof. As those skilled in the art will understand, the angle of the adaptor plates 702, 704 may be selected to widen/reduce a clipping area of the device 700.

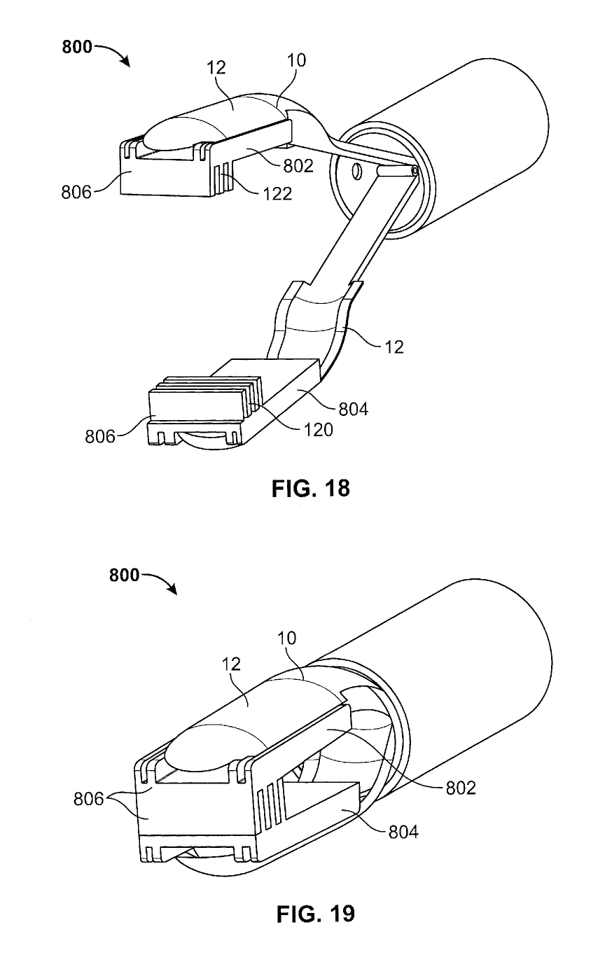

FIGS. 18-19 depict a device 800 according to another embodiment of the invention, the device 800 being formed substantially similarly to the device 100 with the exception of a distal tissue gripping feature provided thereon. Specifically, adaptor plates 802, 804 of the device 800 are positioned over a clip 20 having arms 22 that extend substantially parallel to one another. The arms 22 do not comprise sharpened jaws or teeth extending therefrom. Rather, the exemplary clip 20 for use with the device 800 is configured to reduce trauma to the tissue in an operative configuration. The adaptor plates 802, 804 are removably or permanently attached to the clip 20, as described in greater detail in earlier embodiments. A distal end of a tissue-gripping surface of each adaptor plate comprises an abutment 806 configured to apply a constrictive pressure to a blood vessel or another gripped structure in the body. Each of the abutments 806 comprises protrusions 120, 122 to permit an interlocking engagement thereof, as described in greater detail earlier. The protrusions 120, 122 may be provided over tissue-gripping surfaces of the abutments 806 in any configuration without deviating from the scope of the invention (e.g., parallel to a longitudinal axis of the clip 20, perpendicular to the longitudinal axis of the clip 20 or at any other angle). It is further noted that the protrusions may be replaced by any other surface without deviating from the scope of the invention (e.g., smooth, roughened, etc.).

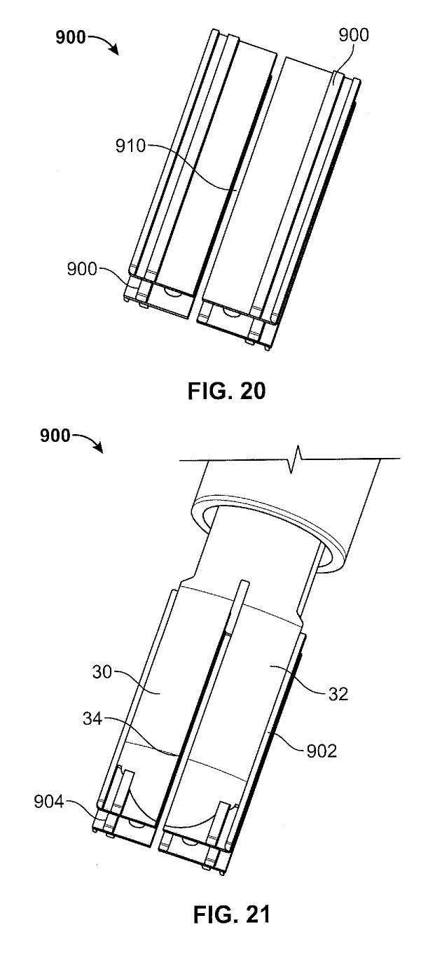

FIGS. 20-21 depict a device 900 according to another embodiment of the invention, the device 900 being formed substantially similar to the device 100 with the exception of a longitudinal slot 910 extending through adaptor plates 902, 904. Specifically, a clip 30 housing the adaptor plates 902, 904 may comprise a slot 34 extending through first and second arms 32 thereof. The slot 910 of the adaptor plates 902, 904 is configured to align with the slot 36 in an operative configuration. The first and second adaptor plates 902, 904 may be configured to interlock with one another in the manner disclosed earlier (i.e., snap-fit, etc.). In another embodiment (not shown), a polymer or other material may be embedded in the slot 910 to lock halves of each adaptor plate 902, 904 to one another. The polymer may be formed of a material permitting a scalpel or other device to cut therethrough when performing a medical procedure. Specifically, in operation, the clip 30 with the attached adaptor plates 902, 904 may be positioned over a vessel or other lumen to cause a constriction thereof. A cutting device (e.g., a scalpel, scissors, etc.) may be inserted through the slots 36, 910 to permit cutting of the vessel. In yet another embodiment (not shown), the clip 900 may comprise a blade or other cutting element disposed in place of the slots so that closure of the clip over the tissue also causes automatically causes a clipping thereof.

In yet another embodiment of the invention (not shown), the adaptor plates may be formed with a snap-fit design, ratcheting snaps or symmetrical parts wherein the first and second adaptor plates may be identical to one another.

It will be understood by those of skill in the art that individual features of the embodiments described above may be omitted and or combined to form alternate embodiments. Furthermore, it will be understood by those skilled in the art that various modifications can be made in the structure and the methodology of the present invention, without departing from the spirit or scope of the invention. For example, any portion of any of the devices disclosed herein may include a sheath mounted thereover. Furthermore, in some embodiments, the clips may be integral with the adaptor plates or may be formed separately. The clips and adaptor plates may be formed of the same or different materials. The adaptor plates may be treated to enhance a gripping quality thereof. The adaptor plates may further comprise a non-slip coating (e.g., silicone, etc.), a soft layer provided over the protrusions and may be formed with surface features (e.g., small teeth, bumps, notches, surface roughening, etc.) In another embodiment, a soft material may be used to form the adaptor plates, as those skilled in the art will understand. In another embodiment, the exemplary device disclosed herein may be used to grasp tissue for dissection, manipulation, retraction, and for any other temporary or permanent need. Thus, it is intended that the present invention cover the modifications and variations of this invention provided that they come within the scope of the appended claims and their equivalents.

What is claimed is:

1. A system for performing a medical operation, comprising:
an end effector having first and second arms, each extending from a distal end to a proximal end, the proximal ends being connected to one another, the end effector being movable between a closed configuration wherein the first and second arms are separated from one another by a first distance and an open configuration wherein the first and second arms are separated from one another by a second distance greater than the first distance, a distance between the first and second arms being substantially constant from the proximal ends to the distal ends, wherein the end effector is biased to the open configuration;
a first plate element extending from a first surface configured to engage a first wall of the first arm to a second surface configured to contact target tissue in an operative configuration; and
a second plate element extending from a first surface configured to engage a first wall of the second jaw to a second surface configured to contact target tissue in an operative configuration.

2. The system of claim 1, wherein the proximal ends of each arm are integral to one another.

3. The system of claim 1, wherein the end effector includes a clipping device.

4. The system of claim 1, further comprising a capsule housing a proximal portion of the end effector therewithin.

5. The system of claim 1, further comprising a first protrusion on the second surface of the first plate element.

6. The system of claim 5, further comprising a second protrusion on the second surface of the second plate element.

7. The system of claim 6, wherein the second protrusion is configured to interlink with the first protrusion to apply a constrictive pressure to target tissue captured therebetween when the end effector is in the closed configuration.

8. The system of claim 7, further comprising a recess formed on the second surface of the second plate element, the recess being configured to receive the first protrusion therein to apply a constrictive pressure to target tissue captured therebetween when the end effector is in the closed configuration.

9. The system of claim 1, wherein the first surface of the first plate element comprises a first groove configured to lockingly engage an extension portion of the first arm and the first surface of the second plate element comprises a second groove configured to lockingly engage an extension portion of the second arm.

10. The system of claim 1, wherein the first and second plate members are substantially planar.

11. The system of claim 1, further comprising a locking arrangement configured to lock the first plate element to the second plate element in the closed configuration.

12. The system of claim 11, wherein the locking arrangement comprises a tab extending from a distal end of the first plate element, the tab being configured to lockingly grip the first surface of the second plate element when moved to the closed configuration.

13. The system of claim 1, wherein the first plate element is integrally formed with the second plate element.

14. The system of claim 1, wherein the first plate element is connected to the second plate element by a hinge.

15. The system of claim 1, further comprising a slot extending through the first and second plate elements and the end effector, the slot configured to permit insertion of a cutting device therethrough.

16. A medical device, comprising:
a first plate element extending from a first surface configured to engage a first wall of a first arm of an end effector to a second tissue-contacting surface; and
a second plate element extending from a first surface configured to engage a first wall of a second arm of the end effector to a second tissue contacting surface, wherein the first and second arms of the end effector are connected to one another at a proximal end and are movable between a closed configuration wherein the first and second arms are separated from one another by a first distance and an open configuration wherein the first and second arms are separated from one another by a second distance greater than the first distance, a distance between the first and second arms being substantially constant from the proximal ends to the distal ends.

17. The device of claim 16, further comprising a first protrusion on the second surface of the first plate element.

18. The device of claim 17 further comprising a second protrusion on the second surface of the second plate element.

19. The device of claim 18, wherein the second protrusion is configured to interlink with the first protrusion to apply a constrictive pressure to target tissue captured therebetween when the end effector is in the closed configuration.

20. The device of claim 19, further comprising a recess formed on the second surface of the second plate element, the recess being configured to receive the first protrusion therein to apply a constrictive pressure to target tissue captured therebetween when the end effector is in the closed configuration.

21. The device of claim 17, wherein the first protrusion is one of linear, curved, S-shaped, serpentine and zig-zagged.

22. The device of claim 16, wherein the first surface of the first plate element comprises a groove configured to lockingly engage an extension portion of the first jaw.

23. The device of claim 16, wherein the first plate member is substantially planar.

24. The device of claim 16, further comprising a locking arrangement configured to lock the first plate element to the second plate element in the closed configuration.

25. The device of claim 24, wherein the locking arrangement comprises a tab extending from a distal end of the first plate element.

26. The device of claim 25, wherein the tab is configured to lockingly grip the first surface of the second plate element when moved to the closed configuration.

27. The device of claim 16, wherein the first plate element is integrally formed with the second plate element.

28. The device of claim 16, wherein the first plate element is connected to the second plate element by a hinge.

29. The device of claim 16, wherein the first plate element is slidably receivable over the first jaw.

30. The device of claim 29, wherein the first plate element comprises an opening configured to receive the first jaw therethrough.

31. A method for deploying a medical device in a living body, comprising the steps of:
advancing a device to a target position in a living body, the device having a first channel extending longitudinally therethrough and having an end effector positioned therein, the end effector having at least two arms, each extending from a distal end to a proximal end, first and second plates connected to an inner surface of each arm respectively, and configured to contact target tissue in an operative configuration, wherein a distance between the first and second plates is substantially constant along a length thereof;
deploying the end effector at a target location in the body so that one of a blood vessel, duct and an organ is captured between the first and second plate elements; and
moving the end effector to the closed configuration, the movement causing the first and second plate elements to apply a constrictive pressure to the selected one of the blood vessel, duct and organ and prevent a flow of fluid therethrough.
